(19) United States
(12) Patent Application Publication
(10) Pub. No.: US 2016/0331488 A1
(43) Pub. Date: Nov. 17, 2016

(54) MODULE FOR DISPENSING A SUBSTANCE FOR DENTAL USE

(71) Applicants: NSK NAKANISHI INC., Kanuma-shi, Tochigi-Ken (JP); NSK FRANCE, Paris (FR)

(72) Inventor: Herve Noui, Salles d'Aude (FR)

(21) Appl. No.: 15/110,456

(22) PCT Filed: Jan. 7, 2015
(86) PCT No.: PCT/EP2015/050164

§ 371 Date: Jul. 8, 2016

(30) Foreign Application Priority Data
Jan. 8, 2014 (FR) 14 50109

(51) Int. Cl. A61C 1/00; A61M 13/00; A61M 11/02; A61C 3/025; A61C 17/02

(52) U.S. Cl. CPC A61C 1/0084; A61C 3/025; A61C 17/0202; A61M 11/02; A61M 13/00; A61B 2217/007; A61M 2039/248

(57) ABSTRACT

A dispensing device for dispensing a clinical substance for dental or medical use, configured as either integrated into a handpiece for dental or medical use or adjacent to said handpiece. The dispensing device includes at least one exchangeable module comprising a primary fluid inlet, configured to be coupled to a primary fluid supply duct and a fluid outlet, configured to deliver fluid that can be loaded with a clinical substance, the exchangeable module comprising a chamber containing the substance to be dispensed in liquid, gel, or powder form, the chamber forming a substance reserve, such that when primary fluid circulates between inlet and outlet, the fluid can collect clinical substance.

The present invention relates to devices for dispensing a clinical substance and relates particularly to modules for dispensing a clinical substance for applications in the dental or medical field.

More particularly, the invention relates to a device inserted into an assembly of medical and/or dental instruments comprising an applied part, and making it possible to diffuse, within an irrigation liquid, substances intended for clinical or diagnostic purposes. The irrigation liquid in question is usually water, however it can also be a physiological serum. The additive substance to be delivered with the irrigation liquid, can in particular serve to disinfect the clinical site, interrupt bleeding, deliver medicaments, activate a chemical agent, reveal a deposit, etc. A handpiece provided with a dental instrument delivers the irrigation liquid with the additive substance, if appropriate accompanied by a mechanical load such as the production of ultrasonic vibratory movement.

The variety of intended applications requires the use of several distinct additive substances.

It is known in the prior art to have, at the level of the main dental or medical equipment, several reservoirs of fluid, each reservoir comprising irrigation liquid with a particular additive substance. However, given that the reservoirs are separated from the handpiece by a multifunctional flexible line of a certain length, at each change from one reservoir to the other, the multifunctional line and the handpiece should be purged, which results in an unnecessary consumption of fluid, and which represents an additional operation which takes time.

As a result, there is an apparent need to propose a solution in order to improve the above-mentioned dispensing devices, in particular when the use of several different substances one after the other is provided.

To this end, the invention proposes in particular a dispensing device for administrating a substance for dental or medical use, the dispensing device being configured to be either integrated into a handpiece for dental or medical use or adjacent to (or in proximity to) said handpiece,

the dispensing device comprising at least one exchangeable module comprising a primary fluid inlet, configured to be coupled to a primary fluid supply duct and a fluid outlet, configured to deliver fluid that can be loaded with a clinical substance, the exchangeable module comprising a chamber containing the substance to be dispensed under liquid, gel, or powder form, the chamber forming a reserve of substance, such that when a primary fluid circulates between inlet and outlet, the fluid can collect said substance.

Thanks to these arrangements, when it is desired to change the additive substance, it is not necessary to purge the content of the primary fluid supply line. In fact, as the additive substance is introduced into the handpiece or at the vicinity thereof, the primary fluid contains no additive substance upstream of the exchangeable module. Thus, it is sufficient to exchange the exchangeable module in order to change from one additive substance to another, without the need to purge the flexible duct linking the main equipment to the handpiece.

It should be noted here that the handpiece assembly comprising the exchangeable module dispensing the additive substance can also be exchanged.

In embodiments of the device according to the invention, either one or other of the following arrangements can optionally be used.

The exchangeable module can advantageously comprise an axial duct linking the inlet to the outlet, by which means a module exhibiting such an arrangement is suitable for being inserted according to an ‘in-series’ configuration into a dental or medical handpiece or even into an instrument.

The axial duct is preferably connected to the chamber by means of radial passages which allows a flow of the primary fluid through the chamber, preferably selectively and in adjustable manner, so that the additive substance is situated all around the axial duct, and the circulation of the primary fluid allows a certain quantity of substance to be collected when the primary fluid passes into the chamber.

The exchangeable module can also comprise a portion of tube surrounding the axial duct with at least one radial orifice and wherein the axial duct comprises at least one radial orifice, by means of which the portion of tube can serve as a means for controlling the flow rate of the primary fluid inside the chamber through the radial orifices which can be more or less aligned depending on the desired adjustment.

The axial duct can be connected to the chamber by means of axial passages, allowing a natural flow of the primary fluid through the chamber.

The exchangeable module can comprise a control element making it possible to set the flow rate of the primary fluid through the chamber, between a predetermined minimum flow rate or even zero and a predetermined maximum flow rate, so that the practitioner can set the flow rate of substance added to the primary irrigation fluid, in addition to the flow rate of the primary fluid.

The fluid is caused to circulate inside the exchangeable module by pressure which is greater than the atmospheric pressure at the primary fluid inlet. Thus, it is the pressure prevailing in the primary fluid supply duct, upstream of the exchangeable module, which pushes the fluid and its additive in the direction of the clinical site.

The exchangeable module can also comprise a viewing window forming a gauge. Thus, the practitioner can at any moment know the quantity of additive substance present in the exchangeable module.

The exchangeable module can exhibit a generally cylindrical shape and adjustment by rotation about the axis, which represents a particularly optimized shape in order to be able to integrate such a module into a handpiece of generally cylindrical shape or into a dental instrument.

The chamber can comprise a porous or hydrophilic material or also a diffuser. Whereby the substance collection rate varies little as a function of the levels of substance remaining in the module or the cartridge for a given flow rate of irrigation fluid.

The exchangeable module can be arranged on an instrument affixed at the distal end of the handpiece. Whereby the instrument forming a tool can be accompanied by the additive substance and it is very easy to change from one instrument forming a tool to another, allowing the additive substance used to be exchanged at the same time.

The device can also comprise a check valve arranged on the primary fluid supply duct, opened by the insertion of a projecting shape belonging to the exchangeable module and closed by an elastic bias when the projecting shape is pulled out. Whereby, when the exchangeable module is uncoupled from the primary fluid supply duct, the valve is closed and the primary fluid remains in the supply duct without spilling out, and this remains true even if the practitioner presses on the pedal by mistake.

The check valve can be a ball check valve, which represents a simple, reliable and economical solution for forming such a check valve.

The invention also relates to a dental treatment assembly comprising a handpiece, a tool and a dispensing device as described above.

Other characteristics and advantages of the invention will become apparent on reading the following description of several embodiments thereof, given by way of non-limitative examples, with reference to the attached drawings, in which:

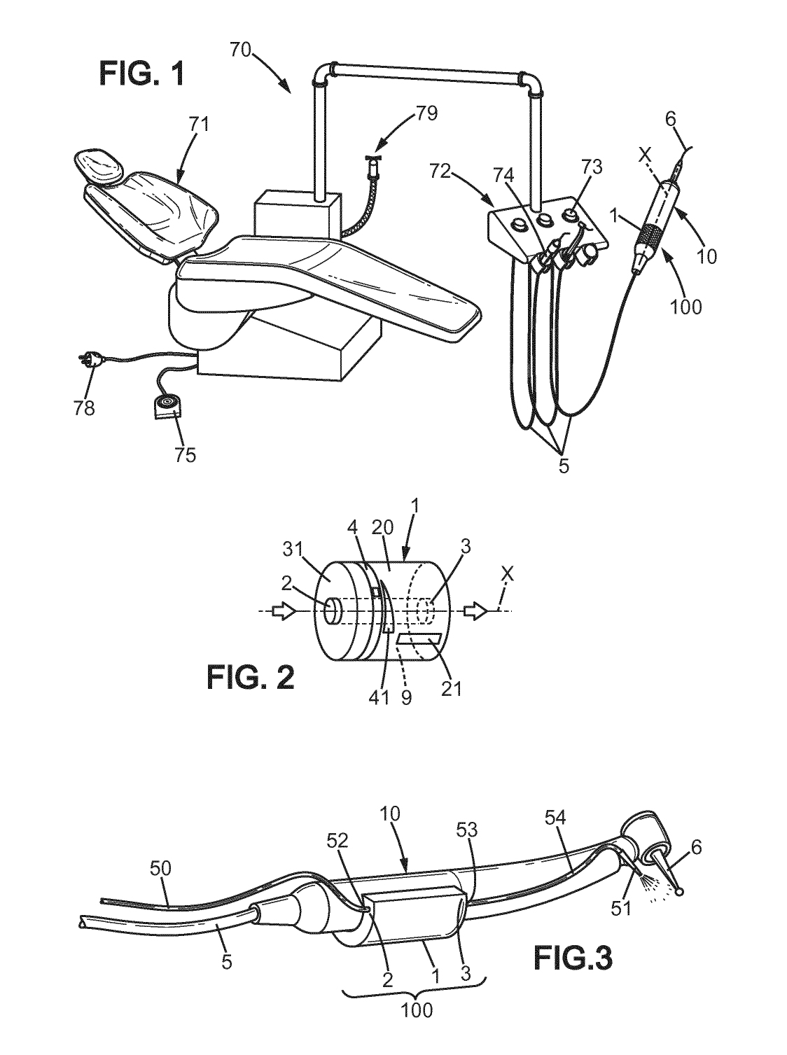

FIG. 1 is a general perspective view of a device according to a first embodiment of the invention,

FIG. 2 represents a diagram of the exchangeable module which can be integrated into a handpiece such as that illustrated in FIG. 1,

FIG. 3 is a general perspective view of a device according to a second embodiment of the invention,

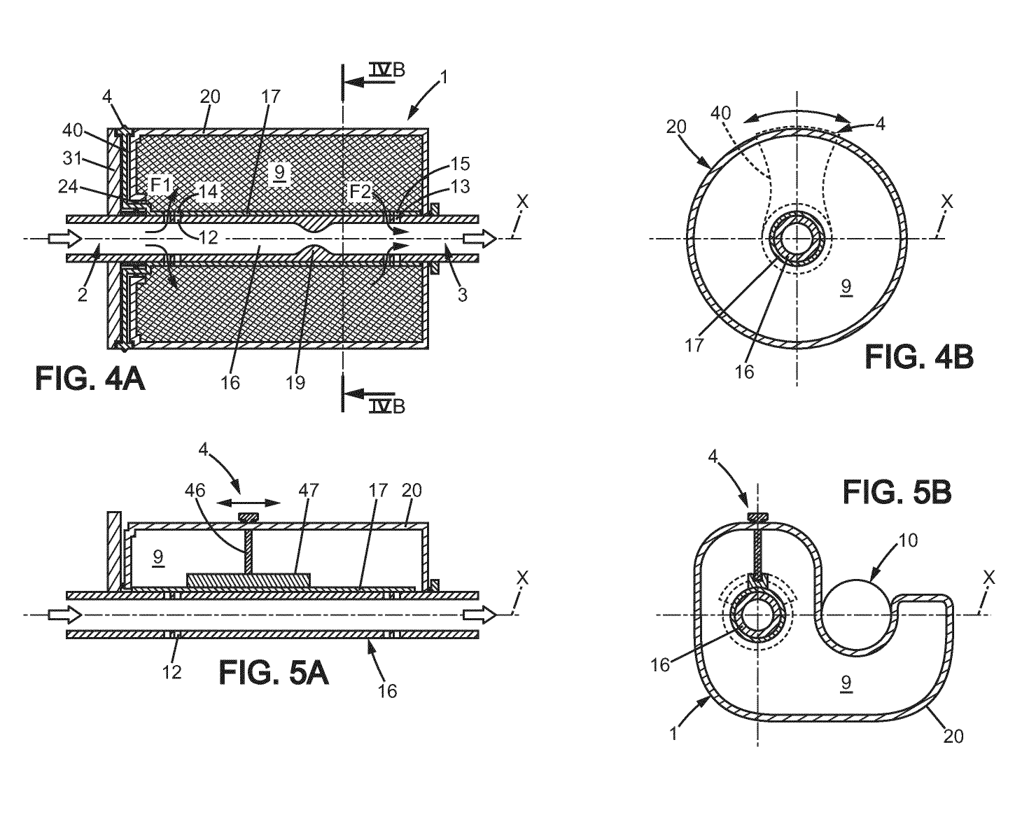

FIGS. 4A and 4B are schematic diagrams showing partial axial-section and cross-section views of the exchangeable module of FIG. 2,

FIGS. 5A and 5B are schematic diagrams of partial axial-section and cross-section views of the exchangeable module which can be integrated into a handpiece such as that illustrated in FIG. 3,

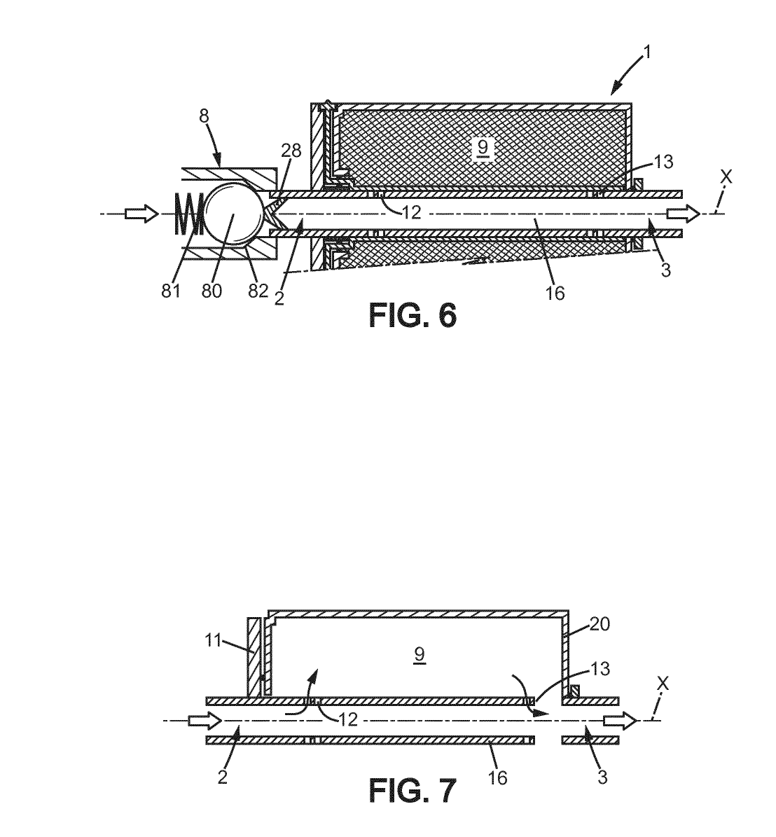

FIG. 6 represents an axial-section view similar to FIG. 4A, in the closed position with, moreover, the coupler of the primary fluid supply duct,

FIG. 7 is similar to FIG. 4A and illustrates another exchangeable module variant,

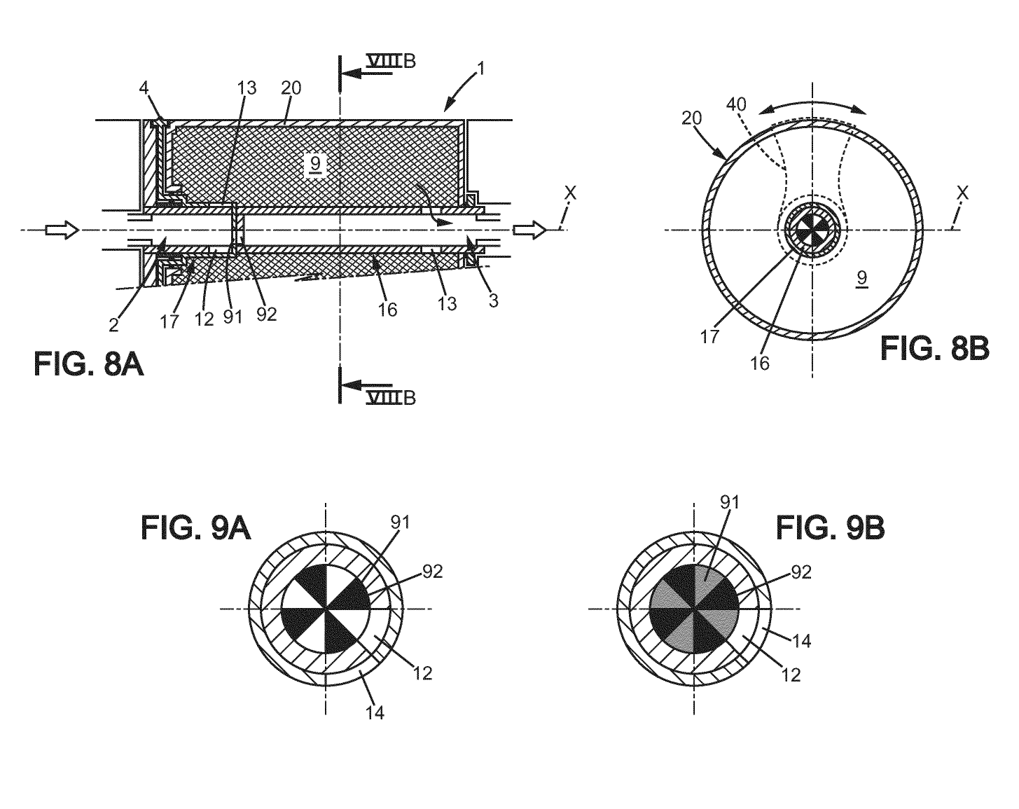

FIGS. 8A and 8B represent a partial axial-section view and a cross-section of an exchangeable module variant of FIG. 2,

FIGS. 9A and 9B are schematic diagrams of detailed cross-section views in the extreme adjustment positions respectively,

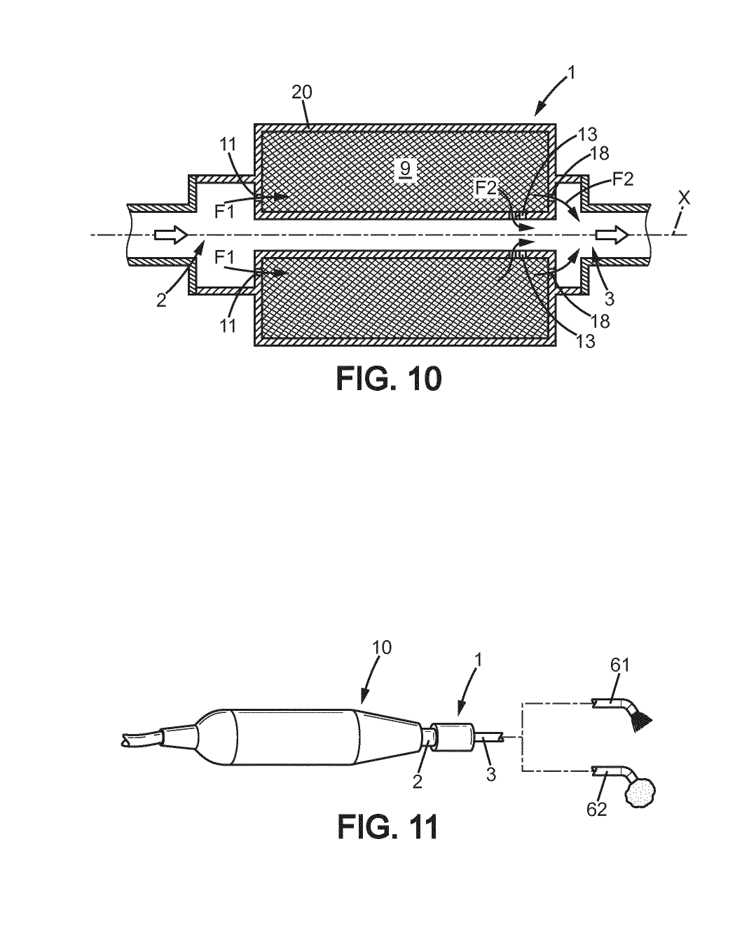

FIG. 10 is a view similar to FIG. 4A, illustrating an embodiment variant with axial passages,

FIG. 11 illustrates an embodiment variant of the first embodiment shown in FIG. 1, with the exchangeable module at the distal end of the handpiece.

In the different figures, the same reference numbers denote identical or similar elements.

FIG. 1 represents a standard installation 70 of a dental practice with a chair 71 which can accommodate a patient, main dental equipment 72, which is generally multifunctional (or several juxtaposed pieces of equipment), connected to handpieces 10, 74 such as a drill and burrs, compressed air nozzle, ultrasonic vibratory tool, etc.

The chair system is connected to the electricity distribution system 78, and can also be connected to the drinking water distribution system 79. The practitioner can use a foot control (pedal 75) and can have various adjustments 73 available on the dental equipment, in particular adjustment of the irrigation liquid flow rate. Each of the handpieces is connected to the dental equipment by means of a multifunctional cable 5, the latter being able to contain compressed air and liquid fluid ducts and electric conductors.

Certain clinical applications use dental instruments 6 requiring the use and routing to the clinical site of an irrigation fluid. The fluid used can serve different needs:
it can allow the basic operating principle of the clinical tool, for example in the case of an ultrasonic instrument used for prophylaxis, the need to have a liquid available in order to generate the cavitation effect intended to remove calcifications that have accumulated on the dental enamel,
it can serve to wash and purge the clinical site of biological tissue fragments generated during treatment,
it can serve to cool the clinical site treated, and prevent any burning and necrosis of biological tissues within the context of use of instruments such as cutting and abrasion tools, the mechanical effect of which generates significant collateral heating,
it can serve to treat biological tissues by dispensing a substance for therapeutic purposes, for example in order to disinfect, interrupt bleeding or deliver medicinal substances, these substances being delivered in the form of solutes routed through the flow of irrigation liquid,
it can make it possible to provide the practitioners, by the application to the clinical site of visible or radio-opaque markers, with visual information for the purposes of diagnosis and location of the zones to be treated.

In the configuration shown in FIG. 1, a device 100 for dispensing an additive substance 9 is provided, this dispensing device being mainly formed by an exchangeable module 1 which is removably inserted into the handpiece 10.

As illustrated in FIG. 2, the exchangeable module 1 can be presented advantageously in the shape of a cylinder with an axial duct having a primary fluid inlet 2 and a fluid outlet 3. The primary fluid inlet is connected to a fluid connector linked to the multifunctional duct 5, whilst the fluid outlet is connected to another fluid connector inside the handpiece in the example illustrated, in fluid communication with the irrigation liquid delivery nozzle close to the tool 6. Advantageously, the exchangeable module 1 can be equipped with a visual gauge device 21 allowing the practitioner to know the quantity of product 9 remaining in the chamber 20.

According to another configuration forming a second embodiment shown in FIG. 3, the cable linking the dental equipment to the handpiece referenced 5 does not contain the primary irrigation fluid duct. On the contrary, the primary irrigation fluid duct is arranged along the main cable 5, and comprises an upstream portion 50 which supplies the exchangeable module 1 and a downstream portion 54 which links the outlet of the exchangeable module to a dispensing nozzle 51 directed towards the clinical site. The exchangeable module is linked to the upstream portion 50 by an upstream connector 52 and to the downstream portion 50 by a downstream connector 53.

In more detailed manner with respect to the exchangeable module configured for the first embodiment, as illustrated in FIGS. 4A, 4B and 6, the exchangeable module 1 comprises a chamber 20 containing the substance 9 to be dispensed, arranged around an axial duct 16 in which the irrigation fluid flows in the direction of the flow arrows F1,F2.

Advantageously, there is an adjustment device 4 which allows the practitioner to dose the quantity of substance which is entrained in the circulation of the primary irrigation fluid. In the present case the adjustment device is formed by a control tube 17 which surrounds the axial duct and is adjacent to the latter, surrounding it. The control tube 17 is linked to a radial control lever 40 and configured in order to rotate about the axis X in relation to the control lever following a predetermined angular course for example between 40 and 80 degrees. Furthermore, a pictogram 41 engraved in the outer body of the module gives an intuitive indication of the possibility of adjustment, and advantageously indicates the current position relative to extreme minimum and maximum values.

In the shown example, the adjustment device 4 is interposed between the enveloppe of the chamber 20 and a front disc 31 rigid with the chamber enveloppe.

It should be noted that such adjustment device can be any control element arragned on the exchangeable module 1 or arranged on the handpiece 10 itself.

The axial duct comprises one or more radial orifices 12 arranged in the upstream portion, namely on the side of the fluid inlet 2, and one or more other radial orifices 13 arranged in the downstream portion, namely on the side of the fluid outlet 3. Moreover, the control tube 17 is equipped, at the same axial positions as the abovementioned radial orifices, with radial orifices or openings 14 corresponding to the upstream radial orifices 12 of the axial duct and with radial orifices or openings 15 corresponding to the downstream radial orifices 13 of the axial duct.

FIG. 4A shows an exemplified case where the radial orifices 12, 13 of the axial duct are aligned with the radial orifices 14, 15 of the control tube 17 which causes a flow of the primary irrigation fluid through the chamber following the arrows illustrated F1, F2, whilst FIG. 6 shows that in another angular position of the control tube, the radial orifices 12, 13 of the axial duct are not aligned with the radial orifices 14, 15 of the control tube. The offset of the radial orifices is adjustable and when they are sufficiently offset, the passage is completely obstructed and the collection of the additive substance is zero.

The radial orifices can be from 1 to 4 in number, or even more, all around the circumference of the axial duct and of the control tube.

It should be noted that the axial duct can optionally be provided with a restriction of the cross-section 19 in order to promote, by creating a loss of hydraulic pressure, the passage of the irrigation fluid to the chamber 20 containing the substance 9.

As known per se, seals 24 are used in order to confer a sealing function on the exchangeable module 1 so as to prevent the irrigation fluid, loaded or not loaded with substance, from flowing out to the outside of the module.

As illustrated in FIG. 6, a check valve 8 is provided on the upstream connector of the primary fluid duct in order that, when the exchangeable module is disconnected from the irrigation fluid inlet, the irrigation fluid is prevented from flowing, in particular in the event of unwanted activation of the irrigation fluid control on the main device for example accidental pressure on the pedal 75. It should be noted that the size of the check valve device 8 has been exaggerated for the sake of clarity, but it can be much smaller in reality.

Advantageously, it is a valve with automatic operation i.e. a valve is returned to the closed position by a spring-type elastic return means, this valve is removed from its seat when the exchangeable module is inserted, which exchangeable module is equipped with a projecting shape 28 which pushes the valve back.

In the example illustrated, the valve is formed by a ball 80 bearing on a seat 82 which is circular or partially spherical in shape and returned to the seat by a standard spiral compression spring 81.

FIG. 7 illustrates a simplified example of the exchangeable module which contains no adjustment element, but simply passage orifices of predetermined shape and cross-section in order to allow the primary irrigation fluid to flow out radially inside the chamber 20 and leaves the chamber towards the axial duct in the vicinity of the fluid outlet. This simplified form of the exchangeable module is particularly suitable for the configuration which is described below (FIG. 10), in which the module is installed in the dental instrument itself.

FIGS. 5A and 5B illustrate another way of designing the exchangeable module, in particular in the second embodiment illustrated in FIG. 3. In this case, the shape of the chamber has a kidney-shaped cross-section and extends along the lower part of the handpiece 10.

In the present case the adjustment control is linear instead of rotary. A control rod 46 is moved parallel to the axis X and causes the rotation of the control tube by means of external spiral threading 47 rotatably rigid with the control tube 17. The operating principle of the radial orifices or openings is identical or similar to that described previously.

FIGS. 8A and 8B illustrate an embodiment variant which makes it possible to obtain any adjustment between full flow and zero flow of the primary fluid flow passing through the axial duct. To this end, the control tube 17 is shorter than in the preceding cases and also comprises a transverse disc provided with four openings 91 in the form of an angular sector alternating with closed sectors and the axial duct (fixed) is also equipped with a transverse wall also equipped with four openings 92 in the form of angular sectors alternating with closed sectors, similar to the shapes present in the disc of the control tube (movable).

In a first extreme adjustment position (FIG. 9A), the openings 91, 92 are facing each other and the fluid passes through the two respective walls of the control tube of the axial duct, so that the fluid can move from the inlet to the outlet directly without passing through the chamber 20 containing the additive substance 9. In fact the radial orifices 12 and 14 are not facing each other in this position. Therefore in this case, the substance collection rate is zero.

In the extreme opposite adjustment position (FIG. 9B), the openings 91, 92 are offset by 45°, and the closed sectors complement each other in order to prevent the fluid from passing in the axial direction inside the axial duct. Conversely, the radial orifices 12, 14 are aligned and allow the fluid to pass from the axial duct to the inside of the chamber 20, and also to return from the chamber towards the axial duct in the vicinity of the fluid outlet via the passage 13, which is permanently opened here. Therefore in this case, the substance collection is maximum.

It should be noted that, as a variant, the control tube 17 could be extended to the right, and have radial orifices 15 as described above.

Besides the extreme positions illustrated, it is understood that, by means of the control lever, it is possible to hold any intermediate position in order to have any desired distribution between direct flow via the axial duct and deviated flow which passes through the chamber 20 containing the substance 9.

FIG. 10 shows an embodiment variant of the exchangeable module 1, with inlet passages 11 arranged axially (and not radially) in the inlet area 2 of the irrigation flow. These axial passages allow irrigation fluid to enter (flow F1) the chamber 20 containing the substance 9 to be dispensed. Similarly in the outlet area 3, axial passages 18 are provided in the transverse wall of the chamber, complementing the radial passages 13, in order to allow the fluid loaded with substance to pass towards the downstream axial duct (flow F2).

In the example illustrated, there is no element for controlling the levels of inclusion of the substance 9. However, a control disc can be provided (not shown) upstream of the wall upstream of the chamber, which can more or less block the inlet passages 11.

FIG. 11 shows a third embodiment in which the exchangeable module is arranged inside a dental or medical instrument; this instrument can have the form of a brush 61, a sponge 62 or any other suitable tool. In this hypothetical case, the dimensions of the exchangeable module 1 are particularly reduced, for example a diameter less than 2 cm and a length less than 4 cm. Moreover, the module can exhibit a additive flow control element as explained above. The module can also besimpler with no flow rate adjustment.

It should be noted that the irrigation fluid can originate from the drinking water system, or from a pre-treated water tank, or also from a physiological serum tank.

1. A dispensing device to dispense a clinical substance for a dental or medical use, the dispensing device being configured to be either integrated into a medical or dental handpiece or adjacent to said handpiece, the dispensing device comprising at least an exchangeable module comprising a primary fluid inlet, configured to be coupled to a primary fluid supply, and a fluid outlet port, configured to deliver fluid that can be loaded by a clinical substance, the exchangeable module comprising a chamber enclosing the clinical substance to be dispensed under liquid, gel, or powder form, the chamber forming a reserve of clinical substance, such that when primary fluid circulates between inlet and outlet, the fluid can collect clinical substance,
wherein there is provided a control member allowing to set the primary fluid flow through the chamber, between zero flow and a predetermined maximum flow.

2. The device according to claim 1, wherein the exchangeable module further comprises an axial duct from the inlet to the outlet port.

3. The device according to claim 2, wherein the axial duct is in fluid communication with the chamber through radial orifices, which allows a flow of primary fluid through the chamber, preferably selectively and in an adjustable manner.

4. The device according to claim 3, further comprising a portion of tube surrounding the axial duct with at least a radial orifice and wherein the axial duct comprises at least a radial orifice.

5. The device according claim 1, wherein the control member is arranged on the exchangeable module.

6. The device according to claim 1, wherein the fluid is caused to circulate into the exchangeable module by the pressure which is greater than the atmospheric pressure at the primary fluid inlet.

7. The device according to claim 1, wherein the exchangeable module further comprises a viewing window forming a gauge.

8. The device according to claim 1, wherein the exchangeable module exhibits a generally cylindrical shape having an axis and an adjustment function by rotation of the control member around the axis.

9. The device according to claim 1, wherein the chamber comprises a porous or hydrophilic material or a diffuser.

10. The device according to claim 1, wherein the exchangeable module is arranged on an instrument affixed at the distal end of the handpiece.

11. The device according to claim 1, further comprising a check valve arranged on the primary fluid supply, opened by the insertion of a projecting shape belonging to the exchangeable module and closed by an elastic bias when the projecting shape is pulled out.

12. The device according to claim 11, wherein the check valve is a ball check valve.

13. The device according to claim 2, wherein the axial duct is in fluid communication with the chamber through axial passages.

14. An assembly for dental treatment comprising a dental handpiece, an instrument and a dispensing device according to claim 1.
